(19) United States
(12) Patent Application Publication
(10) Pub. No.: US 2025/0256700 A1
(43) Pub. Date: Aug. 14, 2025

(54) ELECTRIC ENERGY MANAGEMENT SYSTEM AND METHOD WHILE DRIVING ELECTRIFIED VEHICLE

(71) Applicants: Hyundai Motor Company, Seoul (KR); Kia Corporation, Seoul (KR)

(72) Inventors: Jee Wook HUH, Hwaseong-si (KR); Dong Su HA, Hwaseong-si (KR)

(73) Assignees: Hyundai Motor Company, Seoul (KR); Kia Corporation, Seoul (KR)

(21) Appl. No.: 19/037,589

(22) Filed: Jan. 27, 2025

(30) Foreign Application Priority Data
Oct. 2, 2024 (KR) 10-2024-0133500

(51) Int. Cl. B60W 20/13; A61B 5/16; A61B 5/18; A61B 5/369; B60W 10/06; B60W 10/08; B60W 10/26; B60W 50/14

(52) U.S. Cl. CPC B60W 20/13; A61B 5/163; A61B 5/18; A61B 5/369; B60W 10/06; B60W 10/08; B60W 10/26; B60W 50/14; B60W 2420/403; B60W 2510/0208; B60W 2510/244; B60W 2540/225; B60W 2540/229; B60W 2552/15; B60W 2554/40; B60W 2710/244

(57) ABSTRACT

An electrical energy management method while driving an electrified vehicle, includes entering a stay preparation mode, upon determining that a driver needs to rest and use a stay mode from driver state information obtained by a driver information acquisition device while driving the vehicle, and performing charging control to generate additional charging energy considering vehicle driving energy until the vehicle arrives at a selected stay place through a power generation device configured to generate electrical energy using vehicle fuel and to store the additional charging energy in an energy storage device during the stay preparation mode.

### CROSS-REFERENCE TO RELATED APPLICATION

The present application claims priority to Korean Patent Application No. 10-2024-0133500 filed on Oct. 2, 2024, the entire contents of which is incorporated herein for all purposes by this reference.

### BACKGROUND OF THE PRESENT DISCLOSURE

### Field of the Present Disclosure

The present disclosure relates to an electrical energy management system and method while driving an electrified vehicle configured for executing a stay mode that enables use of electrical devices without worrying about battery charging in the parked state of the vehicle and allows a vehicle user to rest or stay in the vehicle for a long time period.

### Description of Related Art

Recently, as the era of high oil prices continues, the demand for electrified vehicles is increasing, and the proportion of electrified vehicles among all vehicles is expected to gradually increase in the future.

Electrified vehicles may be vehicles that use an electric motor as a driving device to drive the vehicle, and vehicles that use a motor alone or a motor and an engine (i.e., internal combustion engine, ICE) together are known.

Among electrified vehicles, vehicles that use a motor alone include battery electric vehicles (BEVs) that use batteries as a power source (electric power source), and fuel cell electric vehicles (FCEVs) that use fuel cells as a main power source.

Furthermore, vehicles which may use the combined power of a motor and an engine include general hybrid electric vehicles (HEVs) which may charge their batteries only with a self-power generation device, and plug-in hybrid electric vehicles (PHEVs) which may be connected to an external power supply to charge their batteries externally.

Among electrified vehicles, vehicles that can charge their batteries with an external power supply are battery electric vehicles and plug-in hybrid electric vehicles, and vehicles that charge their batteries using power generation devices that use fuel are hybrid electric vehicles (i.e., HEVs and PHEVs) and fuel cell electric vehicles.

In the case of hybrid electric vehicles, an engine that utilizes fuel and a motor (an HSG, a drive motor, or the like) that receives the rotational power of the engine and generates power are power generation devices, and in the case of fuel cell electric vehicles, a fuel cell that utilizes hydrogen as fuel gas is a power generation device.

Among electrified vehicles, battery electric vehicles (BEVs) are called pure electric vehicles, and hybrid electric vehicles (HEVs and PHEVs) that are driven with a motor and fuel cell electric vehicles (FCEVs) may also be called electric vehicles (xEVs) in a broad sense.

In the case of electric vehicles, it is true that there are limitations to market expansion due to charging infrastructure and vehicle prices, but since they may be driven with a motor alone, many users are satisfied with not only quietness and internal comfort but also driving performance.

As the COVID-19 period passes, the lifestyle patterns of vehicle users have changed significantly, the frequency of staying in parked vehicles to rest and use various electrical devices, such as multimedia devices including an audio device, air conditioning devices, electrical products including game consoles and smartphones, and in-vehicle electrical outlets, is gradually increasing, and accordingly, various research and development to further improve internal livability of electrified vehicles is being conducted.

For example, a mode in which a vehicle is electrically connected to an external power supply and utilizes power supplied from the external power supply is known, and separately from the present mode, a mode in which a hybrid electric vehicle (HEV or PHEV) provided with a high-voltage battery idles a self-power generation device, i.e., an engine, to operate a motor or a generator using the rotational power of the engine to generate power for use of electrical devices, such as an air conditioner, is known.

Furthermore, in the case of fuel cell electric vehicles provided with a fuel cell, which is a power generation device that utilizes hydrogen gas as fuel, and a high-voltage battery, a vehicle user may stay in the fuel cell electric vehicle for a long time period while using electrical devices, such as an air conditioner.

However, in the case of hybrid electric vehicles, since idle charging in which a battery is charged by driving an engine may be performed to continuously use electric power, problems, such as noise, vibration, and smoke, may be caused, and problems in terms of engine durability may be caused if the engine is left idling for a long time period.

Therefore, for a vehicle user to rest or stay in the parked state of a vehicle without worrying about battery charging, it is necessary to manage the state of charge (hereinafter referred to as “SOC”) of the battery while driving just before the vehicle reaches a parking space.

However, in the past, there was no known SOC management and control technology for future vehicle stay that manages the SOC value of a battery in advance while a vehicle moves to a parking space to use electricity in the parked state of the vehicle.

Furthermore, in the past, SOC control was mainly used to improve fuel efficiency or drivability, and SOC control for driver convenience has not been known up to now. Furthermore, the reality is that the existing electric field control technology for securing the SOC is optimized only for charging situations.

The information included in this Background of the present disclosure is only for enhancement of understanding of the general background of the present disclosure and may not be taken as an acknowledgement or any form of suggestion that this information forms the prior art already known to a person skilled in the art.

### BRIEF SUMMARY

Various aspects of the present disclosure are directed to providing an electrical energy management system and method while driving a vehicle for a vehicle mode (stay mode) that are applicable to an electrified vehicle configured for executing the stay mode that enables use of electrical devices without worrying about battery charging in the parked state of the vehicle and allows a vehicle user to rest or stay in the vehicle for a long time period.

In one aspect, the present disclosure provides an electrical energy management system while driving an electrified vehicle, including a power generation device provided to generate electrical energy using vehicle fuel, an energy storage device operatively connected to the power generation device and provided to store the electrical energy, a driver information acquisition device provided to obtain driver state information, and a control unit operatively connected to the driver information acquisition device and configured to determine whether a driver needs to rest and use a stay mode from the driver state information obtained by the driver information acquisition device while driving the vehicle, wherein the control unit, upon concluding that the driver needs to rest and use the stay mode, enters a stay preparation mode, and is configured to perform charging control to generate additional charging energy considering vehicle driving energy until the vehicle arrives at a selected stay place through the power generation device and to store the additional charging energy in the energy storage device during the stay preparation mode.

In an exemplary embodiment of the present disclosure, the stay mode may be a mode set to enable use of vehicle energy including the electrical energy stored in the energy storage device while a vehicle user stays in the vehicle in a parked state.

In another exemplary embodiment of the present disclosure, the electrical energy management system may further include an information output device provided to output information, and the control unit may be configured for controlling the information output device to recommend stay places where the stay mode is usable upon concluding that the driver needs to rest and use the stay mode, and may be set to enter the stay preparation mode if the driver selects one of the recommended stay places.

In yet another exemplary embodiment of the present disclosure, the electrical energy management system may further include an input device connected to the control unit and provided to be used by a vehicle user including the driver to input or select information while driving the vehicle, and the control unit may be set to enter the stay preparation mode if the driver selects the one of the recommended stay places through the input device.

In yet another exemplary embodiment of the present disclosure, the control unit may be configured for controlling the information output device to output an alarm message to recommend entry to the stay preparation mode upon concluding that the driver needs to rest and use the stay mode, and may perform control to recommend the stay places if the driver agrees to enter the stay preparation mode through the input device in response to the alarm message.

In still yet another exemplary embodiment of the present disclosure, the electrical energy management system may further include a navigation device connected to the control unit to transmit information related to a vehicle position and stay places around the vehicle to the control unit and guide routes to the stay places, and the control unit may be configured for controlling the information output device to output the information related to the stay places around the vehicle upon concluding that the driver needs to rest and use the stay mode.

In a further exemplary embodiment of the present disclosure, the control unit may receive driving route information including vehicle position information, road slope information to the stay place, and real-time traffic information to the stay place from a navigation device provided in the vehicle, and may be configured to determine vehicle driving energy to the stay place based on the received driving route information.

In another further exemplary embodiment of the present disclosure, the charging control may be control configured to increase a state of charge (SOC) value of a battery, which is the energy storage device, to a target SOC until the vehicle arrives at the stay place.

In yet another further exemplary embodiment of the present disclosure, the target SOC may be set to a maximum allowable SOC value of the battery, and the control unit may be configured to determine additional required energy from an additional required SOC value, which is a difference between a current battery SOC value and the target SOC value, and a battery capacity, and may be configured to determine the additional charging energy from the vehicle driving energy and the additional required energy.

In yet another further exemplary embodiment of the present disclosure, the vehicle may be a hybrid electric vehicle, the charging control may be control configured to lower an EV line configured to determine whether to turn an engine on or off, and the control unit may lower the EV line by a change corresponding to the additional charging energy.

In still yet another further exemplary embodiment of the present disclosure, the vehicle may be a hybrid electric vehicle provided with an engine and a motor as driving devices, and the power generation device may include the engine and at least one of a starter generator connected to the engine to be configured for transmitting power to the engine to start the engine and configured for generating power with rotational power of the engine, or a drive motor configured for generating power with the rotational power of the engine transmitted in an engaged state of an engine clutch.

In a still further exemplary embodiment of the present disclosure, the vehicle may be a fuel cell electric vehicle, and the power generation device is a fuel cell system including a fuel cell configured to generate electrical energy with fuel gas.

In a yet still further exemplary embodiment of the present disclosure, the driver information acquisition device may include a gaze state detector configured to image the driver through a camera and obtain driver's forward gaze state information based on a captured driver image, and a brainwave measuring device configured for measuring driver's brainwaves and obtain driver's driving state information based on the measured brainwave information.

In another further exemplary embodiment of the present disclosure, the control unit may be configured to determine that the driver needs to rest or use the stay mode, if a continuous driving time after the vehicle starts exceeds a set time, if a forward gaze ratio (%) defined as a ratio of a driver's forward gaze time to a set time as the driver's forward gaze state information received from the gaze state detector is less than a predetermined value, or if a signal indicating a drowsy state is received as the driver's driving state information from the brainwave measuring device.

In another aspect, the present disclosure provides an electrical energy management method while driving an electrified vehicle, including determining, by a control unit, whether a driver needs to rest and use a stay mode from driver state information obtained by a driver information acquisition device while driving the vehicle, entering, by the control unit, a stay preparation mode, upon concluding that the driver needs to rest and use the stay mode, and performing, by the control unit, charging control to generate additional charging energy considering vehicle driving energy until the vehicle arrives at a selected stay place through a power generation device configured to generate electrical energy using vehicle fuel and to store the additional charging energy in an energy storage device during the stay preparation mode.

In an exemplary embodiment of the present disclosure, the electrical energy management method may further include controlling, by the control unit, an information output device to recommend stay places where the stay mode is usable, upon concluding that the driver needs to rest and use the stay mode, and the control unit may be set to enter the stay preparation mode if the driver selects one of the recommended stay places.

In another exemplary embodiment of the present disclosure, the charging control may be control configured to increase a state of charge (SOC) value of a battery, which is the energy storage device, to a target SOC until the vehicle arrives at the stay place.

In yet another exemplary embodiment of the present disclosure, the target SOC may be set to a maximum allowable SOC value of the battery, and the control unit may be configured to determine additional required energy from an additional required SOC value, which is a difference between a current battery SOC value and the target SOC value, and a battery capacity, and may be configured to determine the additional charging energy from the vehicle driving energy and the additional required energy.

In yet another exemplary embodiment of the present disclosure, the vehicle may be a hybrid electric vehicle, the charging control may be control configured to lower an EV line configured to determine whether to turn an engine on or off, and the control unit may lower the EV line by a change corresponding to the additional charging energy.

In still yet another exemplary embodiment of the present disclosure, the driver information acquisition device may include a gaze state detector configured to image the driver through a camera and obtain driver's forward gaze state information based on a captured driver image, and a brainwave measuring device configured for measuring driver's brainwaves and obtain driver's driving state information based on the measured brainwave information.

In a further exemplary embodiment of the present disclosure, the control unit may be configured to determine that the driver needs to rest or use the stay mode, if a continuous driving time after the vehicle starts exceeds a set time, if a forward gaze ratio (%) defined as a ratio of a forward gaze time to a set time as the driver's forward gaze state information received from the gaze state detector is less than a predetermined value, or if a signal indicating a drowsy state is received as the driver's driving state information from the brainwave measuring device.

Other aspects and exemplary embodiments of the present disclosure are discussed infra.

The above and other features of the present disclosure are discussed infra.

The methods and apparatuses of the present disclosure have other features and advantages which will be apparent from or are set forth in more detail in the accompanying drawings, which are incorporated herein, and the following Detailed Description, which together serve to explain certain principles of the present disclosure.

### BRIEF DESCRIPTION OF THE DRAWINGS

FIG. 1 is a block diagram showing the configuration of a system of an electrified vehicle to which the present disclosure is applied.

FIG. 2 is a block diagram showing control elements and operating elements that perform a control process for electrical energy management while driving according to an exemplary embodiment of the present disclosure.

FIG. 3 is a flowchart showing the control process of managing electrical energy according to an exemplary embodiment of the present disclosure.

It should be understood that the appended drawings are not necessarily to scale, presenting a somewhat simplified representation of various exemplary features illustrative of the basic principles of the present disclosure. The specific design features of the present disclosure as included herein, including, for example, specific dimensions, orientations, locations, and shapes, will be determined in portion by the particularly intended application and use environment.

In the figures, reference numbers refer to the same or equivalent portions of the present disclosure throughout the several figures of the drawing.

### DETAILED DESCRIPTION

Hereinafter, various exemplary embodiments of the present disclosure will be described in detail with reference to the accompanying drawings. Specific structural or functional descriptions set forth in the exemplary embodiments of the present disclosure will be merely exemplarily provided to describe the exemplary embodiments depending on the concept of the present disclosure, and the exemplary embodiments depending on the concept of the present disclosure may be embodied in different forms. Furthermore, the present disclosure should not be construed as being limited to the exemplary embodiments set forth herein, and it will be understood that the present disclosure includes all modifications, equivalents, or substitutes included in the spirit and technical scope of the present disclosure.

In the following description of the embodiments, terms, such as “first” and “second,” and the like, are used only to describe various elements, and these elements should not be construed as being limited by these terms. These terms are used only to distinguish one element from other elements. For example, a first element described hereinafter may be termed a second element, and similarly, a second element described hereinafter may be termed a first element, without departing from the scope of the present disclosure.

When an element or layer is referred to as being “connected to” or “coupled to” another element or layer, it may be directly connected or coupled to the other element or layer, or intervening elements or layers may be present. In contrast, when an element or layer is referred to as being “directly connected to” or “directly coupled to” another element or layer, there may be no intervening elements or layers present. Other words used to describe relationships between elements should be interpreted in a like fashion, e.g., “between” versus “directly between,” “adjacent” versus “directly adjacent,” etc.

Wherever possible, the same reference numbers will be used throughout the following description to refer to the same or like parts. The terminology used herein is for describing various exemplary embodiments only and is not intended to be limiting. As used herein, singular forms may be intended to include plural forms as well, unless the context clearly indicates otherwise. The terms “includes,” “including,” “including,” and “having” are inclusive and therefore specify the presence of stated features, integers, operations, operations, elements, components, and/or combinations thereof, but do not preclude the presence or addition of one or more other features, integers, operations, operations, elements, components, and/or combinations thereof.

The present disclosure provides an electrical energy management system and method for preparing a vehicle mode while moving to a parking space, which are applicable to an electrified vehicle configured for executing the vehicle mode (stay mode) that enables use of electrical devices without worrying about battery charging in the parked state of the vehicle and allows a vehicle user to rest or stay in the vehicle for a long time period.

The electrified vehicle to which the present disclosure is applied may be a hybrid electric vehicle (HEV or PHEV) or a fuel cell electric vehicle (FCEV) provided with a high-voltage battery, a self-power generation device that generates power using fuel, and a charging device configured to charge the battery with the power generated by the power generation device.

In a hybrid electric vehicle, the power generation device may include an engine which is driven by fuel to generate and provide rotational power, and a motor which may be operated as a generator by receiving the rotational power from the engine. Here, the motor may be a hybrid starter generator (HSG), which is a starter generator, or a drive motor, which is a driving device configured to drive the vehicle.

In a fuel cell hybrid electric vehicle, the power generation device may include a fuel cell that generates electrical energy using hydrogen gas as fuel. In general, a fuel cell for vehicles refers to a fuel cell stack formed by stacking unit cells in a stack form to satisfy a demanded output.

In the fuel cell hybrid electric vehicle, the power generation device may be a fuel cell system including the fuel cell stack, and stack operation devices, such as an air supply device, a hydrogen supply device, and a thermal management system configured to operate the fuel cell stack.

Furthermore, in the hybrid electric vehicle and the fuel cell electric vehicle, the charging device mounted in a vehicle may include a power conversion device, such as an inverter or a converter for charging the high-voltage battery, which is an energy storage device of the vehicle.

The present disclosure aims to provide a comfortable indoor living environment like a battery electric vehicle (BEV), which is a pure electric vehicle, and allow vehicle power and electric devices to be used for as long as possible without worrying about battery charging, in a hybrid electric vehicle (HEV or PHEV) or a fuel cell electric vehicle (FCEV) that includes a self-power generation device as described above and allows a vehicle user, such as a driver, to perform various activities inside the vehicle or outdoors around the vehicle.

In an exemplary embodiment of the present disclosure, as a vehicle mode that allows long term indoor rest or stay of the vehicle user, a mode in which the vehicle user can rest or stay while using electric devices with electric power generated by the self-power generation device of the vehicle using vehicle fuel or battery power charged by the self-power generation device (including battery power charged while driving) will be referred to as a “stay mode”.

The stay mode may be defined as a mode which is set and provided so that vehicle energy including electrical energy stored in the energy storage device (e.g., battery) of the vehicle is usable while the vehicle user stays in the vehicle in the parked state.

Furthermore, in an exemplary embodiment of the present disclosure, a mode in which control for electrical energy management according to an exemplary embodiment of the present disclosure is performed while the vehicle is driving to move to a place where the stay mode will be executed, i.e., a place where the vehicle user is stayable, such as a parking lot, a rest area, or a nap stop, will be referred to as a “stay preparation mode.”

The stay preparation mode is a mode while driving to prepare the stay mode in the parked state of the vehicle, and is a mode in which electrical energy management is performed while the vehicle moves to a space where the stay mode will be used, assuming that the stay mode is performed after parking.

Accordingly, after going through the stay preparation mode in which electrical energy management is performed while driving according to an exemplary embodiment of the present disclosure, when the vehicle reaches a stayable place (i.e., a stay place) input and selected by a driver, the stay mode may be executed.

In the electrified vehicle to which the present disclosure is applied, during the stay mode, an air conditioning device configured to generate a comfortable indoor environment while parked, an infotainment device which provides integrated functions of information and entertainment, and various electric devices and in-vehicle outlets that are provided in or connected to the vehicle may be used.

Furthermore, in the stay mode, engine startup may be minimized while stopped to reduce emotional dissatisfaction issues (such as engine idling noise when switching from the EV mode to the HEV mode), and it is possible to use the vehicle as a rest area where the driver rests in the vehicle while parked, after the driver gets to work early to avoid rush hour.

In the stay mode provided by the electric vehicle in the present way, compared to a general internal combustion engine (ICE) vehicle that performs engine idling for a long time period, vehicle fuel efficiency may be improved, exhaust gas emissions may be reduced, and engine durability may be increased.

From the perspective of vehicle manufacturers, it is possible to alter consumers' perception of hybrid electric vehicles, which are still perceived as closer to general internal combustion engine (ICE) vehicles, as closer to pure electric vehicles, and accordingly, it is possible to more effectively and naturally respond to today's market changes that are occurring in the transition period to electric vehicles.

Conventionally, nothing about battery SOC management and a control method thereof while driving a vehicle that are performed to execute the stay mode was known. However, battery SOC management through energy prediction was known, and this is control for maximization of vehicle fuel efficiency, and control logic and process thereof are complicated and thus not suitable for application for preparing the stay mode for driver convenience.

If the SOC value of a battery is not sufficiently secured while driving the vehicle just before arriving at a place where the stay mode of the vehicle will be executed in the parked state, convenience of the stay mode may be halved, and accordingly, may lower the frequency of driver's stay mode use and may also be a factor in worsening marketability of the vehicle.

Therefore, in an exemplary embodiment of the present disclosure, if the driver wants the stay mode, the vehicle may enter the stay preparation mode to perform control for electrical energy management while driving the vehicle, allowing the driver to use the stay mode more comfortably after parking.

Furthermore, the present disclosure proposes a system and method which may provide convenience in control development through simplification of control logic in electrical energy management while driving for preparation of the stay mode and minimize a sense of difference felt by the driver when performing actual control.

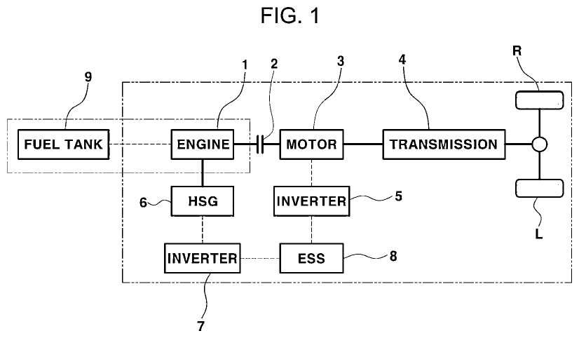

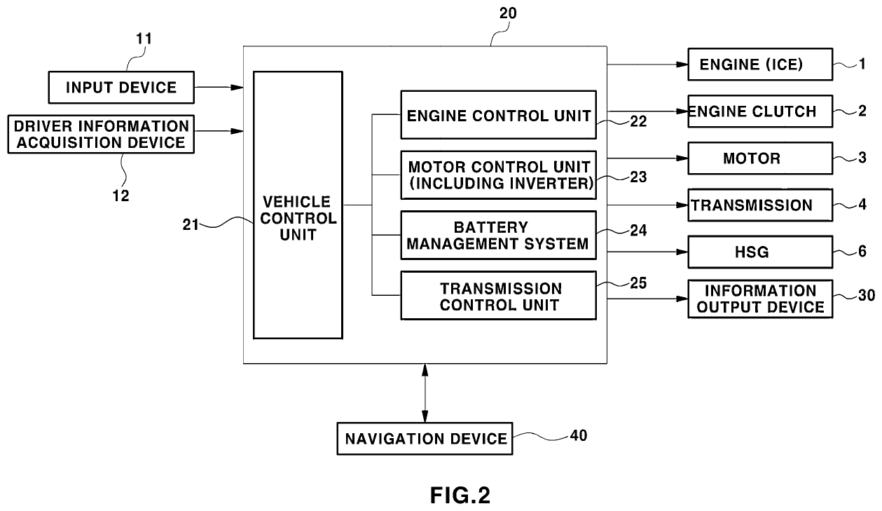

FIG. 1 is a block diagram showing the configuration of a system of an electrified vehicle to which the present disclosure is applied, and FIG. 2 is a block diagram showing control elements and operating elements that perform a control process for electrical energy management while driving according to an exemplary embodiment of the present disclosure.

FIG. 1 illustrates the configuration of a hybrid system including a powertrain apparatus of a parallel hybrid electric vehicle. As shown in the present figure, the hybrid system includes an engine 1 and a motor 3 as driving devices for vehicle driving, an engine clutch 2 located between the engine 1 and the motor 3, a transmission 4 connected to the output side of the motor 3 to be configured for transmitting power to the transmission, an inverter 5 configured to drive the motor 3, and an energy storage system (ESS) 8 connected to the motor 3 through the inverter 5 to be configured for being charged and discharged as a power source (electric power source) of the motor 3.

Here, the motor 3, i.e., a motor, which is the driving device configured to drive the vehicle, is a drive motor mounted as a vehicle driving source in a general hybrid electric vehicle. Furthermore, the ESS 8 may include one or both of a battery and a capacitor.

In the following description, the battery is the ESS 8 which is connected to the self-power generation device of the electrified vehicle to which the present disclosure is applied via inverters 5 and 7, which are charging devices, to be configured for being charged and discharged, and may be a high-voltage battery mounted in a general hybrid electric vehicle.

In FIG. 1, reference numeral “6” indicates a separator motor, i.e., a hybrid starter generator (HSG), which is connected to the engine 1 to be configured for transmitting power to the engine 1 and starts the engine 1 or generates power using rotational power transmitted from the engine 1.

The HSG 6 is operated as a motor or a generator, and is connected to the ESS 8 through the inverter 7 to be configured for being charged and discharged. Furthermore, the HSG 6 is connected to the engine 1 through a power transmission device, such as a belt and a pulley or a gear device, to be configured for transmitting power thereto at all times.

Although FIG. 1 illustrates the hybrid system including the HSG 6 connected to the engine 1 to be configured for transmitting power thereto at all times, the electrified vehicle to which the present disclosure is applied may be a hybrid electric vehicle in which, instead of the HSG 6 described above, a separate motor including a rotation shaft directly connected to the output side of the engine 1 to be configured for transmitting power thereto is mounted.

That is, in the hybrid system illustrated in FIG. 1, the HSG 6 may be removed, and a separate motor directly connected to the output side of the engine 1 to be configured for transmitting power to the engine 1 may be disposed between the engine 1 and the engine clutch 2. The present motor may be used as a motor that starts the engine 1, and may also be used as a generator that assists engine power to drive the vehicle or generates electrical energy using the engine power. The motor on the output side of the engine 1 is also connected to the ESS 8 through an inverter to be configured for being charged and discharged.

The engine clutch 2 is hydraulically engaged (closed) to connect the engine 1 and the motor 3 so that power is transmittable therebetween or disengaged (opened) to separate the engine 1 and the motor 3. In hybrid electric vehicles, while idle charging, the rotational power of the engine 1 may be blocked so as not to be transmitted to the motor 3, and at the instant time, the engine clutch 2 is controlled to separate the engine 1 and the motor 3 to block power transmission.

In the configuration of FIG. 1, each inverter 5 or 7 converts the direct current (DC) of the ESS 8 into three-phase alternating current (AC) to drive the motor 3 or the HSG 6, and supplies the three-phase alternating current to the motor 3 or the HSG 6. On the other hand, if the motor 3 and the HSG 6 are operated to generate power, each inverter 5 or 7 converts the alternating current generated by the motor 3 or the HSG 6 into direct current and supplies the direct current to the ESS 8.

Furthermore, the transmission 4 shifts the power of the motor 3 or the combined power of the engine 1 and the motor 3, and transmits the power to driving wheels L and R through a driveshaft. In FIG. 1, a fuel tank 9 stores fuel to be supplied to the engine 1. Here, the fuel may be a known engine fuel, such as gasoline, diesel, LPG, or the like.

FIG. 1 illustrates the hybrid system, and if the electrified vehicle to which the present disclosure is applied is a hybrid electric vehicle, the power generation device may include the engine 1 that generates and provides rotational power, and a motor that receives the rotational power of the engine 1 and is operated as a generator.

Here, the motor may be at least one of the drive motor 3 and the HSG 6, and the HSG 6 may be operated as a generator with the rotational power of the engine 1 to charge the battery, which is the ESS 8, or if the vehicle is stopped momentarily while driving or is parked, the HSG 6 and the drive motor 3 may be operated as generators with the rotational power of the engine 1 to charge the battery, which is the ESS 8.

Furthermore, any motor which is connected to the engine 1 to receive the rotational power from the engine 1 and is mounted in the vehicle to generate power with the rotational power of the engine 1 and charge the battery may be used as a component of the power generation device together with the engine 1.

If the battery is charged using the HSG 6, the engine clutch 2 is disengaged so that the rotational power of the engine 1 is not transmitted to the drive motor 3, and if the drive motor 3 is used to charge the battery, the transmission 4 is controlled in a state in which power transmission to the output shaft thereof is blocked, i.e., in the neutral (N) state while the engine clutch 2 is engaged.

If the electrified vehicle to which the present disclosure is a fuel cell electric vehicle (FCEV), the fuel tank 9 of FIG. 1 is replaced with a hydrogen tank that stores hydrogen, which is fuel gas, and the power generation device, i.e., the engine 1 and the HSG 6, is replaced with a fuel cell stack.

The vehicle provided with the hybrid system of FIG. 1 may be driven in an electric vehicle (EV) mode, which is a pure electric vehicle mode that utilizes only the power of the motor 3, or in a hybrid electric vehicle (HEV) mode that utilizes the power of the engine 1 and the power of the motor 3 in combination.

Furthermore, when braking the vehicle (when decelerating the vehicle due to brake pedal input) or when decelerating the vehicle by coasting due to inertia, a regenerative mode in which the kinetic energy of the vehicle is recovered as electrical energy through motor generation to charge the battery is performed. Such a regenerative mode function is essential to increase vehicle efficiency and improve fuel efficiency in a hybrid electric vehicle.

Furthermore, the hybrid electric vehicle is provided with a hybrid control unit (HCU) which is also referred to as a vehicle control unit (VCU) 21, which is an upper-level control unit configured to control the overall operation of the vehicle, and various other control units configured to control various devices of the vehicle are provided.

For example, an engine control unit (ECU) 22 that is configured to control the operation of the engine 1, a motor control unit (MCU) 23 that is configured to control the operation of the motor 3, a battery management system (BMS) 24 that collects battery status information, utilizes the information for battery charging and discharging control or provides the information to other control units, and is configured to perform control for managing the battery, which is the ESS 8, and a transmission control unit (TCU) 25 that is configured to control the operation of the transmission 4 may be provided.

The hybrid control unit 21 and the respective control units 22, 23, 24 and 25 perform cooperative control while exchanging information with each other through communication for vehicle power control and driving control, shift control, power generation control, battery charging and discharging control, etc., and the upper-level control unit 21 transmit control commands to the lower-level control units 22, 23, 24 and 25 while collecting various information from the lower-level control units 22, 23, 24 and 25.

The control process for electrical energy management while driving according to an exemplary embodiment of the present disclosure may be performed in the electrified vehicle by a plurality of control units that performs cooperative control as described above, or may be performed by a control unit into which at least some of the control units are integrated, rather than the above-described plurality of control units. For example, the control process may be performed by a vehicle domain control unit (VDCU) into which a plurality of control units including the hybrid control unit HCU are integrated.

Furthermore, the control process may be performed by one control unit including the integrated functions of the control units. Although a control subject has been described above as being divided into the plurality of control units, the control process for electrical energy management while driving according to an exemplary embodiment of the present disclosure may be performed by a single control element which may perform the functions of the plurality of control units integrally, instead of the plurality of control units described above.

In an exemplary embodiment of the present disclosure, the plurality of control units and the single control element may all be collectively referred as a control unit, and the control process according to an exemplary embodiment of the present disclosure, which will be described below, may be performed by the present control unit. In the following description, a control unit 20 collectively refers to the plurality of control units and the single control element.

Furthermore, a control system that is configured to perform electrical energy management according to an exemplary embodiment of the present disclosure includes an input device 11, a driver information acquisition device 12, an information output device 30, and a navigation device 40, in addition to the control unit 20.

The input device 11 is used when the vehicle user (hereinafter including a driver) wants to input necessary information during electrical energy management while driving and a control process thereof according to an exemplary embodiment of the present disclosure. For example, the vehicle user such as the driver may request entry to the stay preparation mode or select entry to the stay preparation mode by operating the input device 11.

The input device 11 is electrically connected to the control unit 20. That is, the input device 11 is connected to the control unit 20 so that an electrical signal may be input through the input device 11, and any unit which is provided in the vehicle to be operated for input and selection, such as a switch, a button, or a touchscreen, may be applied as the input device 11 in an exemplary embodiment of the present disclosure.

For example, the input device 11 may be an input device of an audio, video and navigation (AVN) system or audio, video, navigation and telematics (AVNT) system, and may and may be a touchscreen of the Audio, Video and Navigation (AVN) system or the AVNT system.

Furthermore, the input device 11 may include a mobile device configured for installing and executing an application related to the stay mode. The mobile device may be able to be connected to the control unit 20 in the vehicle, and for the present purpose, an input/output communication interface for communication connection between mobile device and the control unit 20 is used.

Therefore, when an electrical signal depending on operation of the input device 11 is input to the control unit 20, the control unit 20 may recognize that the vehicle user requests or selects entry to the stay preparation mode, and may then enter and start the stay preparation mode.

The information output device 30 outputs various information that needs to be provided to the vehicle user such as the driver during the control process for electrical energy management while driving according to an exemplary embodiment of the present disclosure, and the information output device 30 may include a display device that displays the information, and may further include a sound output device that outputs the information as sound.

Here, the display device may include a display device of a cluster or the navigation device 40. If a cluster is used as the display device, a cluster control unit, which is a control subject that performs communication and cooperative control with other control units, participates in the control process according to an exemplary embodiment of the present disclosure.

The navigation device 40 is electrically connected to the control unit 20 to enable input and output of signals, and is configured to perform a general road guidance function. That is, when the driver inputs and sets a destination in the navigation device 40, the navigation device 40 searches for and generates driving routes to the destination, and is configured to determine the expected driving distance, expected driving time, and expected arrival time for each driving route.

Thereafter, the navigation device 40 displays various navigation information including the driving route on the map selected by the driver, speed limits, the remaining driving distance and remaining driving time to the destination, and the expected arrival time through the display device to provide the navigation information to the driver. Furthermore, the navigation device 40 transmits and inputs destination arrival information that notifies that the vehicle has arrived at the destination to the control unit 20 when the vehicle arrives at the destination.

Furthermore, the navigation device 40 transmits information on stay places around the vehicle where the stay mode is configured for being executed and used, real-time vehicle position information obtained through a built-in Global Positioning System (GPS) receiver, road gradient information to the stay places, and real-time traffic information to the stay places received from outside the vehicle to the control unit 20.

In an exemplary embodiment of the present disclosure, the control unit 20 recommends entry to the stay preparation mode through the information output device 30 if it is determined that the driver needs to rest and use the stay mode based on driver state information obtained through the driver information acquisition device 12.

In an exemplary embodiment of the present disclosure, the driver information acquisition device 12 is configured to obtain the driver state information, and may include a gaze state detector that images of the driver and obtains driver's forward gaze state information based on a captured driver image, and a brainwave measuring device which is configured for measuring driver's brainwaves and obtains driver's driving state information based on the measured brainwave information.

Here, the gaze state detector may include a camera that images the driver, and a determiner that analyzes and is configured to determine a driver's forward gaze state from the driver image captured by the camera and outputs information indicating the forward gaze state (i.e., the driver's forward gaze state information).

In an exemplary embodiment of the present disclosure, the determiner of the gaze state detector may be a processor and a memory.

The camera may be provided in front of the driver's seat in the vehicle to image the driver, and may be provided to capture a driver image including the front of the driver's upper body and a surrounding environment, a driver image including at least the driver's face and a surrounding environment. The camera transmits the captured driver image to the determiner in real time.

In an exemplary embodiment of the present disclosure, the determiner of the gaze state detector is provided to store the driver image received from the camera in real time, analyze and determine the driver's forward gaze state from the received driver image using an image analysis algorithm already widely known in the image recognition field, and output the information indicating the determined driver's forward gaze state.

For example, the image analysis algorithm may extract a plurality of feature points from the driver image, extract a driver's facial contour using a boundary extraction technique based on the plurality of extracted feature points, extract specific information of an internal area of the facial contour based on the extracted facial contour information, and determine whether the driver is gazing at front based on whether there are changes in the specific information. Here, the specific information of the internal area may be information corresponding to the position of the pupils.

In the instant case, information related to a state in which the driver drives the vehicle while gazing at front from the driver's seat may be stored in advance in the determiner and then used, and if the state in which the driver drives the vehicle while gazing at front from the driver's seat is captured in advance by the camera before performing the image analysis algorithm, reference information obtained from the driver image captured at the instant time may be stored and registered in advance by the determiner, and the reference information may be used to determine whether the driver is gazing at front by comparing information extracted from a driver image obtained in real time by the determiner with the reference information.

To explain in more detail, the facial contour is extracted from the driver image information captured when the driver is gazing at front and stored as reference facial contour information, and specific information of the internal area of the reference facial contour, i.e., information corresponding to the position of the pupils, is image-processed and stored as reference pupil information.

The process of capturing the image of the driver when the driver is gazing at front to obtain the reference facial contour information and the reference pupil information may be a process that the driver performs in advance through guidance in the vehicle.

Thereafter, when actual driving starts and the driver image captured by the camera is received in real time by the determiner, the determiner may extract driver's facial contour information and pupil information from the real-time captured driver image, and may be configured to determine whether the driver is gazing at front by comparing the extracted driver's facial contour information and pupil information with the reference facial contour information and reference pupil information to determine whether they match or how different face and gaze angles are from each other.

Alternatively, as an exemplary embodiment of the present disclosure, the image analysis algorithm may be provided to obtain driver's real-time gaze data from the driver image received from the camera, and then determine the driver's forward gaze state based on the obtained driver's gaze data.

Here, the driver's gaze data may include 3D gaze coordinates corresponding to the driver's pupils, and the 3D gaze coordinates may be coordinates including a depth direction (perspective).

In the instant case, driver's gaze data when the driver drives the vehicle while gazing at front from the driver's seat may be stored in advance in the determiner and then used, and the determiner may be configured to determine whether the driver is gazing at front by comparing gaze data obtained in real time from the driver image received from the camera while actually driving with the stored gaze data.

At the present time, if the gaze data while actually driving shows a difference from the stored gaze data by a set level or more, it may be determined that the driver is not gazing at front.

Furthermore, in an exemplary embodiment of the present disclosure, the information indicating the driver's forward gaze state may be a forward gaze ratio, and the forward gaze ratio may be determined based on a time for which the driver does not gaze at front (i.e., a forward non-gaze time).

For example, the forward gaze ratio may be defined as the ratio (%) of a forward gaze time to a preset time. Here, the forward gaze time may be determined by measuring a time for which the driver is gazing at front by the determiner.

In an exemplary embodiment of the present disclosure, the control unit 20 as the determiner of the gaze state detector, may be set to receive the forward gaze ratio determined as described above from the gaze state detector, and to recommend entry to the stay preparation mode through the information output device 30 if the received forward gaze ratio is lower than a predetermined value.

In an exemplary embodiment of the present disclosure, the brainwave measuring device may include a brainwave detection sensor which is configured to detect the driver's brainwaves, and a determiner that is configured to determine the driver's driving state information based on the driver's brainwave information detected in real time by the brainwave detection sensor and outputs the determined driver's driving state information.

In an exemplary embodiment of the present disclosure, the determiner of the brainwave measuring device may be a processor and a memory.

The brainwave detection sensor may be implemented in various forms which may be worn on the driver's head, for example, it may be implemented as a band or a headset type which may be detachably attached to the driver's head.

The brainwave detection sensor may include a plurality of electrode units provide to be in contact with the driver's brain when the brainwave detection sensor is worn by the driver. At least one of the plurality of electrode units may be disposed to detect brainwaves generated from the frontal lobe and occipital lobe of the driver's brain, and at least one other electrode unit may be disposed to detect brainwaves generated from the frontal lobe and occipital lobe of the driver's brain. However, this is an example, and the present disclosure is not necessarily limited thereto.

The determiner of the brainwave measuring device is configured to determine whether the driver's driving state is a drowsy state from the brainwave information detected by the brainwave detection sensor, and outputs a signal indicating the drowsy state as the driver's driving state information to the control unit 20 if it is determined the driver's driving state is the drowsy state.

In an exemplary embodiment of the present disclosure, the control unit 20 as the determiner of the brainwave measuring device, may be set to recommend entry to the stay preparation mode through the information output device 30 if the control unit 20 receives the information indicating the driver's drowsy state from the brainwave measuring device.

A known method may be used as a brainwave analysis method of determining the driver's driving state, whether the driver's driving state is the drowsy state, from the brainwave information detected through the electrode units, as described above.

The method or process of determining whether the driver's driving state is the drowsy state from the brainwave information is already known to those skilled in the art, and a detailed description thereof will thus be omitted herein.

In the present way, the driver's forward gaze state information input from the determiner of the gaze state detector and the driver's driving state information input from the determiner of the brainwave measuring device may be input to the control unit 20.

In an exemplary embodiment of the present disclosure, the control unit 20 as the determiner of the gaze state detector and the brainwave measuring device is configured to determine whether the driver needs to rest and use the stay preparation mode based on the input driver's forward gaze state information and driver's driving state information, and recommends entry to the stay preparation mode through the information output device 30 if it is determined that the driver needs to rest and use the stay preparation mode.

The input device 11, the driver information acquisition device 12, the information output device 30, and the navigation device 40 have been described above, and these devices 11, 12, 30, and 40 are electrically connected to the control unit 20 to input electrical signals to the control unit 20 or receive electrical signals from the control unit 20.

Although FIG. 2 illustrates the input device 11, the information output device 30, and the navigation device 40 as separate components, these devices 11, 30, and 40 may be replaced with an Audio, Video and Navigation (AVN) or AVNT system which is provided in advance in the vehicle and is configured for performing the functions of the devices 11, 30, and 40, and in the instant case, the control unit of the Audio, Video and Navigation (AVN) or AVNT system may be a control subject that performs communication and cooperative control with other control units, and may participate in the control process of the present disclosure.

Hereinafter, an electrical energy management method according to an exemplary embodiment of the present disclosure will be described in detail.

FG. 3 is a flowchart showing the control process of managing electrical energy according to an exemplary embodiment of the present disclosure.

The present disclosure relates to an electrical energy management system and method during the stay preparation mode while driving. To provide the stay mode, a place (hereinafter referred to as a “stay place”) where the vehicle user such as the driver may rest or stay in the vehicle while using electric devices in the vehicle in the parked state is required.

Considering this, in an exemplary embodiment of the present disclosure, the control unit 20 may be set to enter the stay preparation mode if the vehicle user inputs or selects a desired stay place through the input device 11 while the vehicle is driving.

Accordingly, the control unit 20 may be set to perform a thermal energy management process for the stay mode at the stay place during the stay preparation mode while driving.

As a method for the vehicle user such as the driver to determine entry to the stay preparation mode and start of electrical energy management control while driving, a method in which the driver selects and inputs a stay place at his or her own will may be used.

In an exemplary embodiment of the present disclosure, the stay preparation mode includes an active mode in which, if the driver wants to enter the stay preparation mode while driving, the driver requests the vehicle to enter the stay preparation mode, and when the vehicle recommends stay places around the current position of the vehicle, the driver selects one of the recommended stay places, and thereby the vehicle enters the stay preparation mode.

Furthermore, the stay preparation mode includes a passive mode in which, if the vehicle first recommends entry to the stay preparation mode and the driver agrees to enter the stay preparation mode through the input device 11, the vehicle recommends stay places around the current position of the vehicle, the driver selects one of the recommended stay places, and thereby the vehicle enters the stay preparation mode.

In an exemplary embodiment of the present disclosure, if the driver selects one of the active mode and the passive mode through the input device 11, the control unit 20 may allow the vehicle to enter the stay preparation mode in the mode selected by the driver.

In the active mode, if the driver wants to enter the stay preparation mode, the driver may request the control unit 20 of the vehicle to enter the stay preparation mode through the input device 11, and the control unit 20 may output information related to the closest stay place (stayable place) to the current position of the vehicle through the information output device 30 to inform the driver of the information.

Next, as the method for the driver to determine and select entry to the stay preparation mode and start of electrical energy management control, a method in which the driver selects and inputs a stay place at his or her own will may be used.

That is, if the driver inputs the stay place at his or her own will through the input device 11, the control unit 20 enters the stay preparation mode, and starts control for electrical energy management while driving.

In the passive mode, if the vehicle recommends the driver to enter the stay preparation mode through the information output device 30 and recommends stay places, the driver may finally select one of the stay places through the input device 11.

At the present time, if the driver selects one of the stay places through the input device 11, the control unit 20 that has received the selected stay place enters the stay preparation mode, and starts control for electrical energy management while driving.

The control unit 20 may be set to recommend entry to the stay preparation mode through the information output device 30 if the control unit 20 determines that the driver needs to rest and use the stay mode based on the driver state information obtained through the driver information acquisition device 12 in the passive mode.

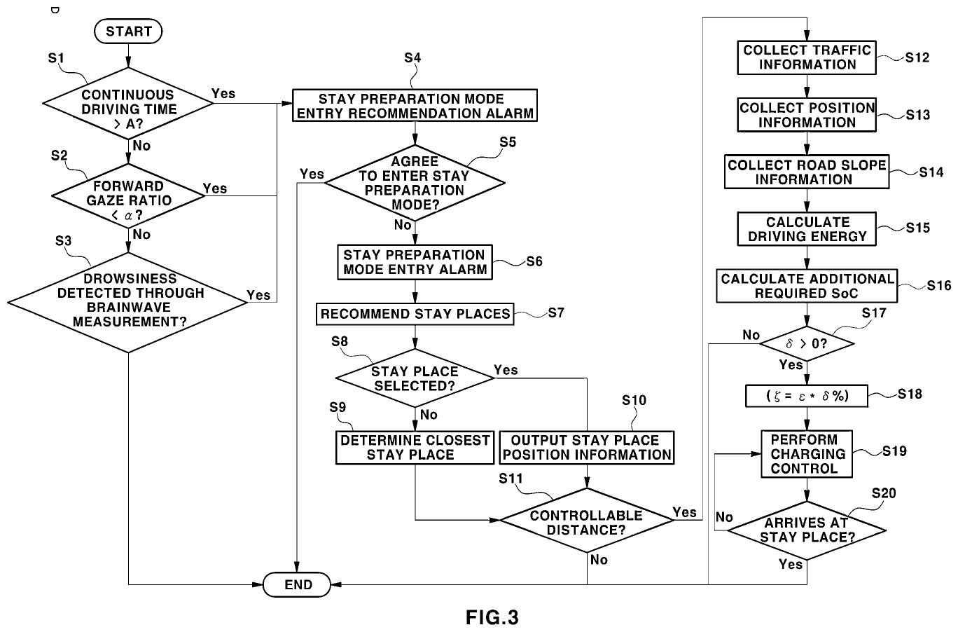

Referring to the flowchart shown in FIG. 3, the control unit 20 may be set to recommend entry to the stay preparation mode if a driver attention warning is required in conjunction with a driver attention warning system.

Here, the driver attention warning may be a warning indicating that a continuous driving time after starting the vehicle exceeds a set time A (Operation S1). That is, if the continuous driving time exceeds the set time A, the control unit 20 may recommend entry to the stay preparation mode through the information output device 30 (Operation S4).

Furthermore, if the forward gaze ratio received from the driver information acquisition device 12 is less than a predetermined value a while the continuous driving time does not exceed the set time A (Yes in Operation S2), the control unit 20 may recommend entry to the stay preparation mode through the information output device 30 (Operation S4).

Furthermore, if the forward gaze ratio received from the driver information acquisition device 12 is greater than or equal to the predetermined value a while the continuous driving time does not exceed the set time A, and the driver's driving state received from the driver information acquisition device 12 is the drowsy state (Yes in Operation S3), the control unit 20 may recommend entry to the stay preparation mode through the information output device 30 (Operation S4).

If the continuous driving time does not exceed the set time A, the forward gaze ratio is greater than or equal to the predetermined value a, and the driver's driving state received from the driver information acquisition device 12 is not the drowsy state, the stay preparation mode is not performed without recommending entry to the stay preparation mode.

When the control unit 20 recommends entry to the stay preparation mode through the information output device 30 as described above, the control unit 20 is configured to control operation of the information output device 30 to output an alarm message recommending entry to the stay preparation mode and asking for the driver to agree to entry to the stay preparation mode.

The alarm message recommending entry to the stay preparation mode is output for informing the driver that rest or sleep is needed and receiving an agreement to entry to the stay preparation mode, and at the instant time, the information output device 30 is controlled by the control unit 20 to display the alarm message.

Thereafter, in Operation S5 of FIG. 3, if the driver inputs an agreement to entry to the stay preparation mode through the input device 11 in response to the alarm message, the control unit 20 outputs a notification message notifying of entry to the stay preparation mode through the information output device 30 (Operation S6).

Thereafter, the control unit 20 displays information related to stayable places, i.e., stay places, such as parking lots, rest areas, or nap stops, through the information output device 30 to recommend the stay places (Operation S7), and accordingly, the driver selects one of the displayed stay places through the input device 11 in Operation S8.

If the driver does not select a stay place within a set time, the closest stay place to the current position of the vehicle may be automatically selected (Operation S9).

At the present time, if route guidance to the destination is being displayed through the navigation device 40, after the stay place has been selected as described above, the navigation device 40 outputs the position information of the stay place selected by the driver (Operation S10), sets the stay place as a stopover on the route to the destination, and then displays route guidance until moving to the stay place, which is the stopover.

Furthermore, the control unit 20 is configured to determine whether a distance to the stay place received through the navigation device 40 and selected by the driver corresponds to a distance for which control for electrical energy management is possible, i.e., a controllable distance (Operation S11).

Here, if the distance to the stay place does not correspond to the controllable distance, for example, if the distance to the stay place is shorter than the controllable distance, the driver may be informed that the stay preparation mode is not configured for being performed through the information output device 30, and the stay preparation mode may not be performed.

On the other hand, if the distance to the stay place corresponds to the controllable distance, the control unit 20 may start control for electrical energy management while driving, and at the instant time, the control unit 20 receives driving route information to the stay place (place where the stay mode will be executed) from the navigation device 40.

In an exemplary embodiment of the present disclosure, if an SOC value which may be increased through general battery SOC control from the current point in time is greater than or equal to a value corresponding to a set ratio (e.g., 10%) of a current SOC value, the distance to the stay place may be set to be determined as corresponding to the controllable distance.

If the SOC value which may be increased through the general battery SOC control from the current point in time is less than the value corresponding to the set ratio, the distance to the stay place may be set to be determined as not corresponding to the controllable distance because the battery SOC control is not performed at a level which may be perceived by the driver.

Furthermore, the stay place is a place where the stay mode will be executed, and is a stayable place selected by the driver or the closest stayable place to the vehicle position at a point in time of entering the stay preparation mode. Furthermore, the stay place becomes the destination while the vehicle is moving to the stay place.

Subsequently, the control unit 20 is configured to determine driving energy while the vehicle is moving to the stay place based on the driving route information received from the navigation device 40. When the driving energy is determined, the total energy required to move to the stay place may be obtained, and when the present value and energy required for charging are given, the battery SOC may be secured and managed by changing an EV line.

To determine the driving energy, the control unit 20 receives the driving route information including vehicle position information, road slope information, and real-time traffic information (Operations S12-S14), and here, the traffic information may include average vehicle speed information of a road ahead. An angular velocity of the vehicle for determining the driving energy may be determined from the vehicle speed.

Furthermore, a remaining distance from the current vehicle position to the stay place, which is the destination, may be determined by receiving the real-time position information of the vehicle as the vehicle position information, and an expected driving time may be determined from the remaining distance to the stay place. Furthermore, the control unit 20 may be configured to determine gradient resistance from the road slope information.

Furthermore, the control unit 20 is configured to determine the driving energy depending on the route based on the above-descried driving route information (Operation S15), and the driving energy may be determined by Equation 1 below.

$$[ Equation 1 ]$$

$$DrivinEnerg \text{?} = {\int }_{0}^{- t} ( ( Airresistan \text{?} + Rollinresistan \text{?} + Gradieresistan ) \text{?} \times Tiredynamiradiu \text{?} \times Angulavelocit ) \text{?} dt$$

$$\text{?} \text{indicates text missing or illegible when filed}$$

Referring to Equation 1, the driving energy may be determined from the expected driving time t, the air resistance, the rolling resistance, the gradient resistance, the tire dynamic radius, which is a unique set value of the vehicle, and the angular velocity.

Determination of the air resistance, the rolling resistance, and the gradient resistance in Equation 1 and determination of the driving energy using Equation 1 are technical matters that have been already known to those skilled in the art, and a detailed description thereof will thus be omitted herein.

In the stay preparation mode in which the electrical energy management while driving according to an exemplary embodiment of the present disclosure is performed, the electrical energy management includes a charging control process in which the battery SOC is increased to a target SOC until the vehicle arrives at the stay place to secure a battery SOC required for the stay mode in advance.

When the electrical energy management including the above-described charging control process is performed while the vehicle is moving to the stay place, disturbance to driver's rest due to engine startup for battery charging may be minimized during the stay mode after arriving at the stay place, and the battery SOC may be secured with high efficiency compared to increasing the battery SOC to the target SOC in the idle state and thus it is advantageous in terms of fuel efficiency.

In an exemplary embodiment of the present disclosure, the target SOC may be set to the maximum allowable SOC value of the battery at a level that does not affect durability of the battery. Since securing the battery SOC as much as possible in advance during the stay preparation mode is advantageous for performing the stay mode thereafter, the target SOC may be determined based on the maximum allowable SOC value of each battery manufacturer.

In an exemplary embodiment of the present disclosure, final additional charging energy is determined using the current battery SOC value, the target SOC value, and driving energy information while moving to the stay place, which is the destination, and for the present purpose, an additional required SOC δ (%) is determined by determining a difference between the current battery SOC value and the target SOC γ (Operation 16).

Thereafter, if the additional required SOC δ (%) is greater than 0, additional required energy ξ considering a battery capacity may be determined (Operations S17 and S18), and when the battery capacity is ε, the additional required energy ξ may be determined by the equation ‘ξ=ε×δ’.

Thereafter, the final additional charging energy is determined from the addition required energy in consideration of the driving energy consumed until the vehicle reaches the stay place, which is the destination, from the current position of the vehicle, and charging control to charge the battery by the final additional charging energy until the vehicle reaches the stay place (Operation S19).

At the present time, the control unit 20 may be configured to determine the final additional charging energy by adding the driving energy and the additional required energy, and may perform charging control based on the determined final additional charging energy. Thereafter, when the vehicle reaches the stay place while the charging control is being performed, the control unit 20 terminates the charging control (Operation S20).

In an exemplary embodiment of the present disclosure, the above-described charging control is performed when the additional required SOC δ (%) is greater than 0 in Operation S17, and if not, the vehicle is driven without performing the charging control until the vehicle reaches the stay place.

In an exemplary embodiment of the present disclosure, the charging control performed because the additional required SOC δ (%) is greater than 0 includes control of lowering the EV line. A line that determines whether to turn the engine on or off in a hybrid electric vehicle is referred to as the EV line, and is setting data which is input to and stored in the control unit 20 and is then used to determine whether to turn the engine on or off.

In an exemplary embodiment of the present disclosure, when the charging control starts, the control unit 20 lowers the EV line to secure the SOC value, being configured for securing the additional required energy. When lowering the EV line, the control unit 20 may be configured to determine additional charging energy required during the driving route to the stay place, i.e., a change corresponding to the final additional charging energy determined above, and then lower the EV line by the determined change.

Here, the control unit 20 may use separate setting data, such as a map in which the change is set, as a value corresponding to the additional charging energy.

The EV line may include an engine-on line in which the engine is turned on and an engine-off line in which the engine is turned off depending on driver's required power, and may be generally defined in a map which is determined by the driver's required power depending on the battery SOC value and vehicle speed.

When the engine is turned off from the ON state, the vehicle switches from the HEV mode to the EV mode, and when the engine is turned on from the OFF state, the vehicle switches from the EV mode to the HEV mode.

To explain Furthermore, the EV line specifies the transition conditions from the EV mode to the HEV mode, and a boundary line on a graph where the EV mode and the HEV mode are mutually switched for various variables may be defined as the EV line.

The conditions for switching from the EV mode to the HEV mode may include various variables, and lowering the EV line means adjusting the EV line so that the transition to the HEV mode occurs under lower variable conditions.

One example of the EV line may be a boundary line connecting map values for each vehicle speed and SOC where mutual switching between the EV mode and the HEV mode occurs in a power map where the driver's required power is mapped depending on vehicle speed and SOC.

Here, lowering the EV line may mean lowering conditions for switching from the EV mode to the HEV mode, i.e., lowering the power value for each vehicle speed and SOC where the engine starts.

By lowering the EV line accordingly, SOC defense to maintain the battery SOC higher than the idle charging SOC while the engine is repeatedly turned on or off may be performed.

At the present time, while driving the vehicle after starting the engine, some of engine power is used to generate power (used to charge the battery and manage the SOC), and the remainder is used to drive the vehicle.

The case in which the vehicle is a hybrid electric vehicle has been described above, and if the vehicle is a fuel cell electric vehicle, a fuel cell stack is operated by driving a stack driving device of a fuel cell system so that a battery SOC becomes a target SOC until the vehicle arrives at a stay place.

At the present time, final additional charging energy required to reach the target SOC when arriving at the stay place is determined in consideration of driving energy to the stay place, and then the fuel cell system, which is a self-power generation device, is driven to generate the determined final additional charging energy while the vehicle is driving.

Accordingly, the electrical energy management system and method while driving the electrified vehicle according to an exemplary embodiment of the present disclosure have been described in detail. According to an exemplary embodiment of the present disclosure described above, since optimal battery SOC control in consideration of a driving environment is performed while the vehicle is moving to a stay place, the stay mode may be used without worrying about battery charging in the vehicle after arriving at the stay place.

Furthermore, it is possible to use electric devices without worrying about battery charging, and the stay mode that allows long-term indoor rest and stay of a vehicle user is provided, being configured for satisfying consumer demands for the vehicle and improving vehicle marketability.

Furthermore, since control for minimizing idle charging during the stay mode. i.e., control of the stay preparation mode, is performed before entering the stay mode, and then the vehicle enters the stay mode, it is possible to improve efficiency of the hybrid system and fuel efficiency.

Conventionally, energy waste due to idle charging during the stay mode was great, but according to an exemplary embodiment of the present disclosure, energy waste may be minimized, and fuel efficiency may be improved because the ON state of the engine in the stay mode may be minimized.

As is apparent from the above description, according to an electrical energy management system and method while driving an electrified vehicle according to an exemplary embodiment of the present disclosure, since optimal battery SOC control in consideration of a driving environment is performed while the vehicle is moving to a stay place, a stay mode may be used without worrying about battery charging in the vehicle after arriving at the stay place.

Furthermore, since control for minimizing idle charging during the stay mode. i.e., control of a stay preparation mode, is performed before entering the stay mode, and then the vehicle enters the stay mode, it is possible to improve efficiency of a hybrid system and fuel efficiency.

Conventionally, energy waste due to idle charging during the stay mode was great, but according to an exemplary embodiment of the present disclosure, energy waste may be minimized, and fuel efficiency may be improved because the ON state of an engine in the stay mode may be minimized.

Furthermore, the term related to a control device such as “controller”, “control apparatus”, “control unit”, “control device”, “control module”, “control circuit”, or “server”, etc refers to a hardware device including a memory and a processor configured to execute one or more steps interpreted as an algorithm structure. The memory stores algorithm steps, and the processor executes the algorithm steps to perform one or more processes of a method in accordance with various exemplary embodiments of the present disclosure. The control device according to exemplary embodiments of the present disclosure may be implemented through a nonvolatile memory configured to store algorithms for controlling operation of various components of a vehicle or data about software commands for executing the algorithms, and a processor configured to perform operation to be described above using the data stored in the memory. The memory and the processor may be individual chips. Alternatively, the memory and the processor may be integrated in a single chip. The processor may be implemented as one or more processors. The processor may include various logic circuits and operation circuits, may be configured for processing data according to a program provided from the memory, and may be configured to generate a control signal according to the processing result.

The control device may be at least one microprocessor operated by a predetermined program which may include a series of commands for carrying out the method included in the aforementioned various exemplary embodiments of the present disclosure.

The aforementioned invention can also be embodied as computer readable codes on a computer readable recording medium. The computer readable recording medium is any data storage device that can store data which may be thereafter read by a computer system and store and execute program instructions which may be thereafter read by a computer system. Examples of the computer readable recording medium include Hard Disk Drive (HDD), solid state disk (SSD), Silicon Disk Drive (SDD), read-only memory (ROM), random-access memory (RAM), CD-ROMs, magnetic tapes, floppy discs, optical data storage devices, etc and implementation as carrier waves (e.g., transmission over the Internet). Examples of the program instruction include machine language code such as those generated by a compiler, as well as high-level language code which may be executed by a computer using an interpreter or the like. Furthermore, the non-transitory computer-readable recording medium may be distributed over computer systems connected through a network, and computer-readable program code may be stored and executed in a distributive manner.

In various exemplary embodiments of the present disclosure, each operation described above may be performed by a control device, and the control device may be configured by a plurality of control devices, or an integrated single control device.

In various exemplary embodiments of the present disclosure, the memory and the processor may be provided as one chip, or provided as separate chips.

In various exemplary embodiments of the present disclosure, the scope of the present disclosure includes software or machine-executable commands (e.g., an operating system, an application, firmware, a program, etc.) for enabling operations according to the methods of various embodiments to be executed on an apparatus or a computer, a non-transitory computer-readable medium including such software or commands stored thereon and executable on the apparatus or the computer.

In various exemplary embodiments of the present disclosure, the control device may be implemented in a form of hardware or software, or may be implemented in a combination of hardware and software.

Software implementations may include software components (or elements), object-oriented software components, class components, task components, processes, functions, attributes, procedures, subroutines, program code segments, drivers, firmware, microcode, data, database, data structures, tables, arrays, and variables. The software, data, and the like may be stored in memory and executed by a processor. The memory or processor may employ a variety of means well-known to a person including ordinary knowledge in the art.

Furthermore, the terms such as “unit”, “module”, etc. included in the specification mean units for processing at least one function or operation, which may be implemented by hardware, software, or a combination thereof.

In the flowchart described with reference to the drawings, the flowchart may be performed by the controller or the processor. The order of operations in the flowchart may be changed, a plurality of operations may be merged, or any operation may be divided, and a specific operation may not be performed. Furthermore, the operations in the flowchart may be performed sequentially, but not necessarily performed sequentially. For example, the order of the operations may be changed, and at least two operations may be performed in parallel.

Hereinafter, the fact that pieces of hardware are coupled operatively may include the fact that a direct and/or indirect connection between the pieces of hardware is established by wired and/or wirelessly.

In an exemplary embodiment of the present disclosure, the vehicle may be referred to as being based on a concept including various means of transportation. In some cases, the vehicle may be interpreted as being based on a concept including not only various means of land transportation, such as cars, motorcycles, trucks, and buses, that drive on roads but also various means of transportation such as airplanes, drones, ships, etc.

For convenience in explanation and accurate definition in the appended claims, the terms “upper”, “lower”, “inner”, “outer”, “up”, “down”, “upwards”, “downwards”, “front”, “rear”, “back”, “inside”, “outside”, “inwardly”, “outwardly”, “interior”, “exterior”, “internal”, “external”, “forwards”, and “backwards” are used to describe features of the exemplary embodiments with reference to the positions of such features as displayed in the figures. It will be further understood that the term “connect” or its derivatives refer both to direct and indirect connection.

The term “and/or” may include a combination of a plurality of related listed items or any of a plurality of related listed items. For example, “A and/or B” includes all three cases such as “A”, “B”, and “A and B”.

In exemplary embodiments of the present disclosure, “at least one of A and B” may refer to “at least one of A or B” or “at least one of combinations of at least one of A and B”. Furthermore, “one or more of A and B” may refer to “one or more of A or B” or “one or more of combinations of one or more of A and B”.

In the present specification, unless stated otherwise, a singular expression includes a plural expression unless the context clearly indicates otherwise.

In the exemplary embodiment of the present disclosure, it should be understood that a term such as “include” or “have” is directed to designate that the features, numbers, steps, operations, elements, parts, or combinations thereof described in the specification are present, and does not preclude the possibility of addition or presence of one or more other features, numbers, steps, operations, elements, parts, or combinations thereof.

According to an exemplary embodiment of the present disclosure, components may be combined with each other to be implemented as one, or some components may be omitted.

The foregoing descriptions of specific exemplary embodiments of the present disclosure have been presented for purposes of illustration and description. They are not intended to be exhaustive or to limit the present disclosure to the precise forms disclosed, and obviously many modifications and variations are possible in light of the above teachings. The exemplary embodiments were chosen and described in order to explain certain principles of the invention and their practical application, to enable others skilled in the art to make and utilize various exemplary embodiments of the present disclosure, as well as various alternatives and modifications thereof. It is intended that the scope of the present disclosure be defined by the Claims appended hereto and their equivalents.

What is claimed is:

1. An electrical energy management system while driving an electrified vehicle, the system comprising:
a power generation device provided to generate electrical energy using vehicle fuel;
an energy storage device operatively connected to the power generation device and provided to store the electrical energy;
a driver information acquisition device provided to obtain driver state information; and
a control unit operatively connected to the driver information acquisition device and configured to determine whether a driver needs to rest and use a stay mode from the driver state information obtained by the driver information acquisition device while driving the vehicle,
wherein the control unit, upon concluding that the driver needs to rest and use the stay mode, enters a stay preparation mode, and is configured to perform charging control to generate additional charging energy considering vehicle driving energy until the vehicle arrives at a selected stay place through the power generation device and to store the additional charging energy in the energy storage device during the stay preparation mode.

2. The electrical energy management system of claim 1, wherein the stay mode is a mode set to enable use of vehicle energy including the electrical energy stored in the energy storage device while a vehicle user stays in the vehicle in a parked state.

3. The electrical energy management system of claim 1, further including:
an information output device provided to output information,
wherein the control unit operatively connected to the information output device is configured to:
control the information output device to recommend stay places where the stay mode is usable upon concluding that the driver needs to rest and use the stay mode; and
enter the stay preparation mode in response that the driver selects one of the recommended stay places.

4. The electrical energy management system of claim 3, further including:
an input device connected to the control unit and provided to be used by a vehicle user including the driver to input or select information while driving the vehicle,
wherein the control unit is configured to enter the stay preparation mode in response that the driver selects the one of the recommended stay places through the input device.

5. The electrical energy management system of claim 4, wherein the control unit is configured to:
control the information output device to output an alarm message to recommend entry to the stay preparation mode upon concluding that the driver needs to rest and use the stay mode; and
perform control to recommend the stay places in response that the driver agrees to enter the stay preparation mode through the input device in response to the alarm message.

6. The electrical energy management system of claim 1, further including:
a navigation device connected to the control unit to transmit information related to a vehicle position and stay places around the vehicle to the control unit and guide routes to the stay places,
wherein the control unit is further configured to control the information output device to output the information related to the stay places around the vehicle upon concluding that the driver needs to rest and use the stay mode.

7. The electrical energy management system of claim 1, wherein the control unit is further configured to:
receive driving route information including vehicle position information, road slope information to the stay place, and real-time traffic information to the stay place from a navigation device provided in the vehicle; and
determine vehicle driving energy to the stay place based on the received driving route information.

8. The electrical energy management system of claim 1, wherein the charging control is control configured to increase a state of charge (SOC) value of a battery, which is the energy storage device, to a target SOC until the vehicle arrives at the stay place.

9. The electrical energy management system of claim 8,
wherein the target SOC is set to a maximum allowable SOC value of the battery, and
wherein the control unit is further configured to:
determine additional required energy from an additional required SOC value, which is a difference between a current battery SOC value and the target SOC value, and a battery capacity; and
determine the additional charging energy from the vehicle driving energy and the additional required energy.

10. The electrical energy management system of claim 1,
wherein the vehicle is a hybrid electric vehicle,
wherein the charging control is control configured to lower an EV line configured to determine whether to turn an engine of the vehicle on or off, and
wherein the control unit is further configured to lower the EV line by a change corresponding to the additional charging energy.

11. The electrical energy management system of claim 1,
wherein the vehicle is a hybrid electric vehicle provided with an engine and a motor as driving devices, and
wherein the power generation device includes:
the engine; and
at least one of a starter generator connected to the engine to be configured for transmitting power to the engine to start the engine and configured for generating power with rotational power of the engine, or a drive motor configured for generating power with the rotational power of the engine transmitted in an engaged state of an engine clutch.

12. The electrical energy management system of claim 1, wherein the vehicle is a fuel cell electric vehicle, and the power generation device is a fuel cell system including a fuel cell configured to generate the electrical energy with fuel gas.

13. The electrical energy management system of claim 1, wherein the driver information acquisition device includes:
a gaze state detector configured to image the driver through a camera and obtain driver's forward gaze state information based on a captured driver image; and
a brainwave measuring device configured for measuring driver's brainwaves and obtain driver's driving state information based on the measured brainwave information.

14. The electrical energy management system of claim 13, wherein the control unit is further configured to determine that the driver needs to rest or use the stay mode:
in response that a continuous driving time after the vehicle starts exceeds a set time;
in response that a forward gaze ratio (%) defined as a ratio of a driver's forward gaze time to a set time as the driver's forward gaze state information received from the gaze state detector is less than a predetermined value; or
in response that a signal indicating a drowsy state is received as the driver's driving state information from the brainwave measuring device.

15. An electrical energy management method while driving an electrified vehicle, the method comprising:
determining, by a control unit, whether a driver needs to rest and use a stay mode from driver state information obtained by a driver information acquisition device operatively connected to the control unit, while driving the vehicle;
entering, by the control unit, a stay preparation mode, upon concluding that the driver needs to rest and use the stay mode; and
performing, by the control unit, charging control to generate additional charging energy considering vehicle driving energy until the vehicle arrives at a selected stay place through a power generation device configured to generate electrical energy using vehicle fuel and to store the additional charging energy in an energy storage device during the stay preparation mode.

16. The electrical energy management method of claim 15, further including:
controlling, by the control unit, an information output device operatively connected to the control unit to recommend stay places where the stay mode is usable, upon concluding that the driver needs to rest and use the stay mode,
wherein the control unit is further configured to enter the stay preparation mode in response that the driver selects one of the recommended stay places.

17. The electrical energy management method of claim 15, wherein the charging control is control configured to increase a state of charge (SOC) value of a battery, which is the energy storage device, to a target SOC until the vehicle arrives at the stay place.

18. The electrical energy management method of claim 17,
wherein the target SOC is configured to a maximum allowable SOC value of the battery, and
wherein the control unit is further configured to:
determine the additional required energy from an additional required SOC value, which is a difference between a current battery SOC value and a target SOC value, and a battery capacity; and
determine the additional charging energy from the vehicle driving energy and the additional required energy.

19. The electrical energy management method of claim 15,
wherein the vehicle is a hybrid electric vehicle,
wherein the charging control is control configured to lower an EV line configured to determine whether to turn an engine of the vehicle on or off, and
wherein the control unit is further configured to lower the EV line by a change corresponding to the additional charging energy.

20. The electrical energy management method of claim 15, wherein the driver information acquisition device includes:
a gaze state detector configured to image the driver through a camera and obtain driver's forward gaze state information based on a captured driver image; and
a brainwave measuring device configured for measuring driver's brainwaves and obtain driver's driving state information based on the measured brainwave information.
